(19) United States
(12) Patent Application Publication
(10) Pub. No.: US 2011/0297883 A1
(43) Pub. Date: Dec. 8, 2011

(54) Compositions of esters of fluorosubstituted alcanoic acids

(75) Inventor: Max Braun, Wedemark (DE)

(73) Assignee: SOLVAY FLUOR GMBH, Hannover (DE)

(21) Appl. No.: 13/201,988

(22) PCT Filed: Feb. 18, 2010
(86) PCT No.: PCT/EP2010/052067

§ 371 Date: Aug. 17, 2011

Related U.S. Application Data
(60) Provisional application No. 61/153,897, filed on Feb. 19, 2009.

(51) Int. Cl. C09K 3/00; C07C 69/66

(52) U.S. Cl. 252/364; 560/174

(57) ABSTRACT

Composition of esters of fluorosubstituted alcanoic acids, comprising or consisting essentially of a compound of a formula selected from the group consisting of:
RCFClC(OAc)═CHC(O)OR¹ (II); RCFHC(O)CH₂C(O)OR¹ (IV);
RCFHC(OAc)═CHC(O)OR¹ (V); and RCFHCH(OAc)CH₂C(O)OR¹ (VI); or of compounds of formula (I) RCFClC(O)CH₂C(O)OR¹ and of formula (II); of compounds of formulae (IV) and (V); or of compounds of formulae (IV) and (VI); wherein R is C₂F₅, CF₃ or F and R¹ is an alkyl group with from 1 to 4 carbon atoms, an alkyl group with from 1 to 4 carbon atoms substituted by 1 or more fluorine atoms. A process for the reduction of the compound of formula (I) and/or formula (II), and compositions resulting from such reduction. A process for the separation of the compound of formula (I) from the compound of formula (II) comprising subjecting a composition comprising such compounds to a distillation operation.

The present application claims the benefit of U.S. application No. 61/153897, filed Feb. 19, 2009, the whole content of which being herein incorporated by reference.

The invention concerns composition of esters of fluorosubstituted alcanoic acids which can be obtained, for example, by addition of fluorochloroalkyl carboxylic acid chlorides to ketene followed by esterification.

Esters of 4,4-difluoro-3-oxo-butanoic acid, especially the ethyl ester, are useful as building blocks in chemical synthesis. For example, these esters are useful for preparing 3-difluoromethyl-4-pyrazole-carboxylic acid esters which are intermediates for the manufacture of pyrazole carboxanilide fungicides. The preparation of such fungicides is described in U.S. Pat. No. 5,498,624.

Esters of 4-fluorosubstituted 3-oxo-alcanoic acids, for example, 5,5,5-trifluoro-4-fluoro-3-oxo-pentanoic acid esters, are suitable as solvents.

Known methods to prepare esters of 4,4-difluoro-3-oxo-alcanoic acid are described in the following by the example of esters of 4,4-difluoro-3-oxo-butanoic acid. The methyl and ethyl esters of 4,4-difluoro-3-oxo-butanoic acid can be prepared by condensation with acetic acid esters under basic conditions. An alternative route is described in EP-A0 694526. According to that reference, polyfluorocarboxylic acid chlorides or anhydrides are reacted with a carboxylic acid chloride in the presence of a tertiary amine, e.g. pyridine. Then, esterification is performed with an alcohol, for example, methanol or ethanol.

It is an object of the present invention to provide new building blocks useful in the chemical synthesis of new products. This object is achieved by the invention as outlined in the claims.

The present invention concerns in particular compositions comprising a compound of formula (I)

RCFClC(O)CH₂C(O)OR¹ (I)

and a compound of formula (II)

RCFClC(OAc)═CHC(O)OR¹ (II)

wherein R is C₂F₅, CF₃ or F and R¹ is an alkyl group with from 1 to 4 carbon atoms, an alkyl group with from 1 to 4 carbon atoms substituted by 1 or more fluorine atoms. R preferably represents F. R¹ preferably represents methyl, ethyl, n-propyl or i-propyl. It is particularly preferred if R¹ represents ethyl.

Such composition can be obtained, for example, by reacting a compound of formula (III)

RCFClC(O)X (III)

wherein R is C₂F₅, CF₃ or F and X is a leaving group, preferably halogen and more preferably Cl, with ketene thereby forming a first reaction product comprising at least addition products of ketene and the compound of formula (III) and subjecting at least part of said addition products to an esterification step.

The reaction can be carried out under the conditions described in PCT application WO 2009/021987 the content of which is incorporated herein by reference into the present patent application.

It has been found that the molar ratio between the compound of formula (III) and ketene influences the molar ratio between compound of formula (I) and compound of formula (II) in the reaction product. By controlling the amount of ketene provided to the reaction medium, it is possible to enhance the yield of desired compound (I) or (II) respectively.

In a first embodiment, the molar ratio between compound of formula (III) and ketene is from 1:0.95 to 1:3.5, preferably from 1:1 to 1:3. This embodiment allows to obtain compositions according to the invention having a molar ratio between the compound of formula (I) and the compound of formula (II) which is from 1:0.01 to 1:0.5, preferably from 1:0.1 to 1:0.2.

In another embodiment, the molar ratio between compound of formula (III) and ketene is from more than 1:3.5 to 1:5, preferably from 1:4 to 1:5. This embodiment allows to obtain compositions according to the invention having a molar ratio between the compound of formula (I) and the compound of formula (II) which is from 0.01:1 to 0.5:1, preferably from 0.01:1 to 0.2.

In another embodiment, the compound of formula (II) can be produced by reacting a compound of formula (I) with ketene, in particular under conditions described herein for the reaction of compound of formula (III) with ketene.

The addition step can be performed in the gas phase or in the liquid phase. Preferably, the pressure is selected so that the gaseous ketene is introduced into compound of formula (III) present in a liquid phase. The temperature is preferably in the range of −50° C. to +60° C., most preferably in the range of −30° C. to +10° C. Preferably, the pressure corresponds to the ambient pressure, but it can be higher than ambient pressure. Preferably, the pressure is equal to or lower than 5 bars (abs).

If desired, the addition reaction can be performed in an aprotic organic solvent, for example, in an aliphatic or aromatic hydrocarbon, or a halogenated hydrocarbon, e.g. in a chlorinated hydrocarbon such as chloroform or dichloromethane. Good results were obtained with dichloromethane.

The esterification can be performed in any known manner. A very simple embodiment provides for the reaction of the acid chloride with the respective alcohol in the absence or the presence of a base.

The esterification step is preferably performed in the liquid phase. Preferably, the pressure is equal to ambient pressure. The pressure also may be above ambient pressure, e.g. up to 5 bars (abs). The temperature is preferably in the range of −50° C. to +5° C., most preferably in the range of −30° C. to +5° C.

The molar ratio between the acid chloride and the alcohol preferably lies in a range from 1:0.8 to 1:2.0.

If desired, the addition reaction can be promoted by bases, for example, tertiary amines. If a base is added, it is advisable to cool the reaction mixture. Alternatively, the esterification can be performed in the presence of onium salts as described in U.S. Pat. Nos. 6,525,213 and 5,405,991. The advantage of this kind of reaction is that an ester phase may separate which makes isolation very easy. If no base is applied, it is advantageous to remove HCl which is a reaction product from the reaction mixture. This can be achieved by applying reduced pressure, passing inert gas through the reaction mixture, for example, nitrogen, argon or even dry air, or by heating the reaction mixture.

The invention further relates to a method of using the compound of formula (II) as intermediate in a reaction to form a further compound, such as for example to form a cyclic fluorocompound. In some embodiments, R in formula (II) may be F and/or R¹ in formula (II) may be methyl, ethyl or propyl, preferably ethyl.

The invention also relates to a process for the separation of the compound of formula (I) from the compound of formula (II) which comprises subjecting a composition comprising these compounds of formulae (I) and (II) to a distillation operation. For example, solvent can be removed from a reaction mixture obtained by reaction of ketene with compound of formula (III) dissolved in a solvent by a first distillation, for example under reduced pressure, and concentrated product material obtained from the first distillation can be subjected to a second distillation which is preferably a fractionated distillation so as to recover from said second distillation at least a fraction enriched in respectively, compound of formula (I) or (II).

The invention also relates to a process for the reduction of the compound of formula (I) and/or formula (II).

In a first embodiment, the reduction process according to the invention comprises reacting any of said compounds or their composition with zinc in the presence of an alcohol, preferably the alcohol applied in the optional esterification step. For example, the reaction can be performed as described in WO 2005/085173 with metallic zinc. An alcohol is suitably present as proton source. Advantageously, the alcohol corresponds to the alcohol of the ester group of the compound of formula (I) or (II), in particular as described herein. The alcohol may be present in a molar ratio relative to the sum of moles of compounds of formula (I) and (II) of at least 1, often at least 2. In one particular aspect, the alcohol is used as solvent for the reaction with zinc.

In a second particular embodiment, the reduction process according to the invention comprises reacting any of said compounds or their composition with hydrogen in the presence of a hydrogenation catalyst. Suitable hydrogenation catalysts are for example based on group VIII metals such as platinum and palladium which are preferably supported on a suitable support material, for example carbon in particular active carbon or charcoal. An example of a suitable catalyst comprises palladium on carbon support.

In a first aspect of the reduction process according to the invention, the reduction is substantially limited to substituting halogen, in particular chlorine atom, by a hydrogen atom while present double bonds remain substantially unaffected, thereby forming a composition comprising a compound of formula (IV)

RCFHC(O)CH₂C(O)OR¹ (IV)

wherein R is C₂F₅, CF₃ or F and R¹ is an alkyl group with from 1 to 4 carbon atoms, an alkyl group with from 1 to 4 carbon atoms substituted by 1 or more fluorine atoms
and a compound of formula (V)

RCFHC(OAc)═CHC(O)OR¹ (V)

wherein R is C₂F₅, CF₃ or F and R¹ is an alkyl group with from 1 to 4 carbon atoms, an alkyl group with from 1 to 4 carbon atoms substituted by 1 or more fluorine atoms.

In a second aspect of the reduction process according to the invention, the reduction process comprises substituting halogen, in particular chlorine atom by a hydrogen and simultaneously hydrogenating double bonds thereby forming a composition comprising a compound of formula (IV)

RCFHC(O)CH₂C(O)OR¹ (IV)

wherein R is C₂F₅, CF₃ or F and R¹ is an alkyl group with from 1 to 4 carbon atoms, an alkyl group with from 1 to 4 carbon atoms substituted by 1 or more fluorine atoms
and a compound of formula (VI)

RCFHCH(OAc)CH₂C(O)OR¹ (VI)

wherein R is C₂F₅, CF₃ or F and R¹ is an alkyl group with from 1 to 4 carbon atoms, an alkyl group with from 1 to 4 carbon atoms substituted by 1 or more fluorine atoms.

The invention also concerns the compositions comprising or consisting essentially of compounds of formulae (IV) and (V) or compounds of formulae (IV) and (VI), respectively. In said compositions, the molar ratio between compounds (IV) and (V) or between compounds (IV) and (VI) respectively is preferably as described above for the compositions comprising or consisting essentially of compounds of formulae (I) and (II).

The invention also concerns compounds (V) and (VI) which can be used as intermediates in chemical synthesis.

In a most preferred aspect of the invention described herein, compound (I) is an 4,4-difluoro-4-chloro-3-oxo-butanoic acid ester, in particular the ethyl ester and compound (II) is an 4,4-difluoro-4-chloro-3-O-acetyl-butanoic acid ester, in particular the ethyl ester.

This composition can be obtained from the reaction of difluorochloroacetyl chloride as compound (III) with ketene. In this especially preferred process of the present invention, difluorochloroacetylchloride is reacted with ketene to form a reaction mixture which is preferably directly introduced into the esterification step without isolation of the intermediate products, such as in particular 4,4-difluoro-4-chloro-3-oxobutanoyl chloride and -4,4-difluoro-4-chloro-3-O-acetyl-butanoyl chloride. The invention also concerns the latter acid chlorides.

In this most preferred aspect of the invention, the resulting reaction mixture after esterification can suitably be subjected to a distillation process.

In this case, a first distillation step, at a pressure of from more than 35 mbar to at most 300 mbar and a temperature which is preferably in the range of +20° C. to +30° C. may be carried out. This first distillation step may suitably be followed by at least a second distillation step, at a pressure of preferably at most 35 mbar, and a temperature which is preferably in the range of +30° C. to less than about +65° C. allowing e.g. to recover ethyl-4,4-difluoro-4-chloro3-oxo-butanoic acid. Thereafter the temperature in the second distillation step may be raised to preferably at least to +65° C. to recover e.g. ethyl-4,4-difluoro-4-chloro-3-O-acetyl-butanoic acid.

The difluorochloroacetyl chloride which is applied in the most preferred aspect of the invention is a commercial product. A preferred method to produce it comprises a step of photochemical oxidation of 1,1-difluoro-1,2,2-trichloroethane with oxygen in the presence or absence of promoters of the reaction, for example, chlorine. According to U.S. Pat. No. 5,545,298, the photo oxidation can be performed in the absence of chlorine under irradiation through quartz glass. If desired, the reaction can be performed without pressurization. According to U.S. Pat. No. 5,569,782 photo oxidation is performed in the absence of chlorine under exposure with light of a wavelength equal to or shorter than 290 nm. The undesired wavelengths can be cut off by applying borosilicate glass. Alternatively, radiation sources could be applied which emit radiation essentially only in the desired range. If desired, the oxidation reaction could be performed under unpressurized conditions. The reaction can also be performed under pressure. Fluorinated carboxylic acid chlorides which are alpha-substituted by a chlorine atom can be prepared analogously from respective starting compounds.

The chlorofluorosubstituted starting compounds needed for the photo oxidation reaction can be prepared according to known methods. For example, 1,1,-difluoro-1,2,2-dichloroethane is commercially available; it can be prepared by the reaction of tetrachloroethylene and HF in the presence of catalysts, e.g. tantalum halides or antimony halides, especially antimony (V) chloride or its fluorination products.

The following example is intended to further explain the invention without limiting it.

### EXAMPLE

### Preparation of ethyl-4,4-difluoro-4-chloro 3-oxo-butanoic acid and ethyl-4,4-difluoro-4-chloro-3-O-acetyl- butanoic acid

In a three-neck round bottom flask, chlorodifluoroacetyl chloride (148.92 g, 1 mol) was dissolved in methylene chloride (500 mL) and the solution was cooled to −30° C. During 2 hours, ketene from a ketene generator (at a rate of ca. 930 mmol/h) was passed through the solution of chlorodifluoroacetyl chloride.

The reaction mixture was warmed up to 0° C. and kept for 1 hour at 0° C. Ethanol (61.98 g, 1.94 mol) was added dropwise to the solution while keeping the temperature below 5° C. The solution was stirred for another 0.5 hour. The reaction mixture was transferred to a 2-liter flask and concentrated on a rotary evaporator under reduced pressure (30° C., 300 mBar). The residue (282.78 g) was further distilled over a 60-cm Vigreux column under a pressure of 30 mBar. Ethyl-4,4-difluoro-4-chloro 3-oxo-butanoic acid was recovered at a temperature of 58-65° C. as a colorless liquid. The yield was 85% of the theoretical yield, and a purity of 98.0% was obtained. Ethyl-4,4-difluoro-4-chloro-3-O-acetyl-butanoic acid was recovered at a temperature above 65° C.

1. A composition comprising a compound of formula (I)
RCFClC(O)CH₂C(O)OR¹ (I)
and a compound of formula (II)
RCFClC(OAc)═CHC(O)OR¹ (II)
wherein R is selected from the group consisting of C₂F₅, CF₃, and F; and wherein R¹ is selected from the group consisting of an alkyl group with from 1 to 4 carbon atoms, and an alkyl group with from 1 to 4 carbon atoms substituted by at least one fluorine atom.

2. The composition according to claim 1, wherein R is F.

3. The composition according to claim 1, wherein R¹ is methyl, ethyl or propyl.

4. The composition according to claim 1, wherein the molar ratio between said compound of formula (I) and said compound of formula (II) is from 1:0.02 to 1:0.18.

5. The composition according to claim 1, which consists essentially of the compounds of formulae (I) and (II).

6. A compound of formula (II)
RCFClC(OAc)═CHC(O)OR¹ (II),
wherein R is selected from the group consisting of C₂F₅, CF₃, and F; and wherein R¹ is selected from the group consisting of an alkyl group with from 1 to 4 carbon atoms, and an alkyl group with from 1 to 4 carbon atoms substituted by at least one fluorine atom.

7. A composition consisting essentially of a compound of formula (II)
RCFClC(OAc)═CHC(O)OR¹ (II),
wherein R is selected from the group consisting of C₂F₅, CF₃, and F; and wherein R¹ is selected from the group consisting of an alkyl group with from 1 to 4 carbon atoms, and an alkyl group with from 1 to 4 carbon atoms substituted by at least one fluorine atom.

8. A process for the separation of the compound of formula (I) from the compound of formula (II) as described in claim 1, which comprises subjecting the composition according to claim 1 to a distillation operation.

9. A method for forming a further compound, comprising using a compound of formula (II)
RCFClC(OAc)═CHC(O)OR¹ (II)
wherein R is selected from the group consisting of C₂F₅, CF₃, and F; and wherein R¹ is selected from the group consisting of an alkyl group with from 1 to 4 carbon atoms, and an alkyl group with from 1 to 4 carbon atoms substituted by at least one fluorine atom,
as intermediate in a reaction to form said further compound.

10. The method according to claim 9, wherein R is F.

11. The method according to claim 9, wherein R¹ is selected from the group consisting of methyl, ethyl, and propyl.

12. The method according to claim 9, wherein the formed further compound is a cyclic fluorocompound.

13. A process for the reduction of a compound of formula (I) and/or a compound of formula (II):
RCFClC(O)CH₂C(O)OR¹ (I)
RCFClC(OAc)═CHC(O)OR¹ (II)
wherein R is selected from the group consisting of C₂F₅ CF₃, and F; and wherein R¹ is selected from the group consisting of an alkyl group with from 1 to 4 carbon atoms and an alkyl group with from 1 to 4 carbon atoms substituted by at least one fluorine atom,
said process comprising: reacting any of said compounds or a composition which comprises both compounds with zinc in the presence of an alcohol.

14. A compound of formula (V)
RCFHC(OAc)═CHC(O)OR¹ (V)
wherein R is selected from the group consisting of C₂F₅, CF₃, and F; and wherein R¹ is selected from the group consisting of an alkyl group with from 1 to 4 carbon atoms, and an alkyl group with from 1 to 4 carbon atoms substituted by at least one fluorine atom.

15. The compound according to claim 14, wherein R is F.

16. The compound according to claim 15, wherein R¹ is selected from the group consisting of methyl, ethyl, and propyl.

17. A compound of formula (VI)
RCFHCH(OAc)CH₂C(O)OR¹ (VI)
wherein R is selected from the group consisting of C₂F₅, CF₃, and F; and wherein R¹ is selected from the group consisting of an alkyl group with from 1 to 4 carbon atoms, and an alkyl group with from 1 to 4 carbon atoms substituted by at least one fluorine atom.

18. The compound according to claim 17, wherein R is F.

19. The compound according to claim 17, wherein R¹ is selected from the group consisting of methyl, ethyl, and propyl.

20. A compound of formula (IV)
RCFHC(O)CH₂C(O)OR¹ (IV)
wherein R is selected from the group consisting of C₂F₅, CF₃, and F; and wherein R¹ is selected from the group consisting of an alkyl group with from 1 to 4 carbon atoms, and an alkyl group with from 1 to 4 carbon atoms substituted by at least one fluorine atom.

21. The compound according to claim 20, wherein R is F.

22. The compound according to claim 20, wherein R¹ is selected from the group consisting of methyl, ethyl, and propyl.
